SEQUENCE LISTING (85 sequences)

SEQ ID NO: 1 - protein, 7 aa, Artificial Sequence
  Synthetic
AVPIAQK

SEQ ID NO: 2 - protein, 7 aa, Artificial Sequence
  Synthetic
ATPFQEG

SEQ ID NO: 3 - protein, 7 aa, Artificial Sequence
  Synthetic
AKPLALT

SEQ ID NO: 4 - protein, 7 aa, Artificial Sequence
  Synthetic
AHPGMPQ

SEQ ID NO: 5 - protein, 7 aa, Artificial Sequence
  Synthetic
ATPWVDQ

SEQ ID NO: 6 - protein, 7 aa, Artificial Sequence
  Synthetic
ARPFATY

SEQ ID NO: 7 - protein, 7 aa, Artificial Sequence
  Synthetic
AHPVMPQ

SEQ ID NO: 8 - protein, 7 aa, Artificial Sequence
  Synthetic
EMRLGLE

SEQ ID NO: 9 - protein, 7 aa, Artificial Sequence
  Synthetic
AVPLSTQ

SEQ ID NO: 10 - protein, 7 aa, Artificial Sequence
  Synthetic
LSGANST

SEQ ID NO: 11 - protein, 7 aa, Artificial Sequence
  Synthetic
ARPFSSP

SEQ ID NO: 12 - protein, 7 aa, Artificial Sequence
  Synthetic
ARPLSNI

SEQ ID NO: 13 - protein, 7 aa, Artificial Sequence
  Synthetic
ALPLSHV

SEQ ID NO: 14 - protein, 7 aa, Artificial Sequence
  Synthetic
ATPVFDL

SEQ ID NO: 15 - protein, 7 aa, Artificial Sequence
  Synthetic
AKPFPSA

SEQ ID NO: 16 - protein, 7 aa, Artificial Sequence
  Synthetic
ATPIWLP

SEQ ID NO: 17 - protein, 7 aa, Artificial Sequence
  Synthetic
ANPFLSD

SEQ ID NO: 18 - protein, 7 aa, Artificial Sequence
  Synthetic
AMPYAPG

SEQ ID NO: 19 - protein, 7 aa, Artificial Sequence
  Synthetic
ATSFHDA

SEQ ID NO: 20 - protein, 7 aa, Artificial Sequence
  Synthetic
ALPLTQV

SEQ ID NO: 21 - protein, 7 aa, Artificial Sequence
  Synthetic
TGASHAP

SEQ ID NO: 22 - protein, 7 aa, Artificial Sequence
  VARIANT 5, 6, 7: Xaa = Any Amino Acid
  VARIANT 2: Xaa = Arg, Lys, or His
  VARIANT 4: Xaa = Leu, Ile, or Phe
  Synthetic
AXPXXXX

SEQ ID NO: 23 - protein, 7 aa, Artificial Sequence
  VARIANT 5, 6, 7: Xaa = Any Amino Acid
  VARIANT 4: Xaa = Leu or Phe
  synthetic
ATPXXXX

SEQ ID NO: 24 - protein, 7 aa, Artificial Sequence
  Synthetic
AVPIAQK

SEQ ID NO: 25 - protein, 7 aa, Artificial Sequence
  Synthetic
ATPFQEG

SEQ ID NO: 26 - protein, 7 aa, Artificial Sequence
  Synthetic
AEIFWLP

SEQ ID NO: 27 - protein, 7 aa, Artificial Sequence
  Synthetic
AIPIATS

SEQ ID NO: 28 - protein, 7 aa, Artificial Sequence
  Synthetic
AKPWSPK

SEQ ID NO: 29 - protein, 7 aa, Artificial Sequence
  Synthetic
ANPIPRS

SEQ ID NO: 30 - protein, 7 aa, Artificial Sequence
  Synthetic
AFPFPSA

SEQ ID NO: 31 - protein, 7 aa, Artificial Sequence
  Synthetic
EVPVRTS

SEQ ID NO: 32 - protein, 7 aa, Artificial Sequence
  Synthetic
AFPFPSA

SEQ ID NO: 33 - protein, 7 aa, Artificial Sequence
  Synthetic
LIPIATS

SEQ ID NO: 34 - protein, 7 aa, Artificial Sequence
  Synthetic
ASPITKT

SEQ ID NO: 35 - protein, 7 aa, Artificial Sequence
  Synthetic
AIPIATS

SEQ ID NO: 36 - protein, 7 aa, Artificial Sequence
  Synthetic
AMPYASP

SEQ ID NO: 37 - protein, 7 aa, Artificial Sequence
  Synthetic
SIKWWTP

SEQ ID NO: 38 - protein, 7 aa, Artificial Sequence
  Synthetic
AIPIATS

SEQ ID NO: 39 - protein, 7 aa, Artificial Sequence
  Synthetic
AIPIATS

SEQ ID NO: 40 - protein, 7 aa, Artificial Sequence
  Synthetic
AFPFPSA

SEQ ID NO: 41 - protein, 7 aa, Artificial Sequence
  Synthetic
AFPFPSA

SEQ ID NO: 42 - protein, 7 aa, Artificial Sequence
  Synthetic
AFPFPSA

SEQ ID NO: 43 - protein, 7 aa, Artificial Sequence
  Synthetic
AKPWHFK

SEQ ID NO: 44 - protein, 7 aa, Artificial Sequence
  VARIANT 5, 6, 7: Xaa = Any Amino Acid
  VARIANT 2: Xaa = Ile, Phe, or Val
  VARIANT 4: Xaa = Ile or Phe
  Synthetic
AXPXXXX

SEQ ID NO: 45 - protein, 7 aa, Artificial Sequence
  Synthetic
AVPIAQK

SEQ ID NO: 46 - protein, 7 aa, Artificial Sequence
  Synthetic
ATPFQEG

SEQ ID NO: 47 - protein, 7 aa, Artificial Sequence
  Synthetic
ATPWVLP

SEQ ID NO: 48 - protein, 7 aa, Artificial Sequence
  Synthetic
AVRFPPM

SEQ ID NO: 49 - protein, 7 aa, Artificial Sequence
  Synthetic
ASRIGTT

SEQ ID NO: 50 - protein, 7 aa, Artificial Sequence
  Synthetic
AMPYLGG

SEQ ID NO: 51 - protein, 7 aa, Artificial Sequence
  Synthetic
AIPIATS

SEQ ID NO: 52 - protein, 7 aa, Artificial Sequence
  Synthetic
AFPVSHN

SEQ ID NO: 53 - protein, 7 aa, Artificial Sequence
  Synthetic
SQPFFPF

SEQ ID NO: 54 - protein, 7 aa, Artificial Sequence
  Synthetic
ASPYTIP

SEQ ID NO: 55 - protein, 7 aa, Artificial Sequence
  Synthetic
ANPIPRS

SEQ ID NO: 56 - protein, 7 aa, Artificial Sequence
  Synthetic
AHPYFAA

SEQ ID NO: 57 - protein, 7 aa, Artificial Sequence
  Synthetic
ANPIPRS

SEQ ID NO: 58 - protein, 7 aa, Artificial Sequence
  Synthetic
ATPWVLP

SEQ ID NO: 59 - protein, 7 aa, Artificial Sequence
  Synthetic
AMPYLGG

SEQ ID NO: 60 - skipped
  Synthetic

SEQ ID NO: 61 - protein, 7 aa, Artificial Sequence
  Synthetic
ATPFMAH

SEQ ID NO: 62 - protein, 7 aa, Artificial Sequence
  Synthetic
AFPVSHN

SEQ ID NO: 63 - protein, 7 aa, Artificial Sequence
  Synthetic
ANPIPRS

SEQ ID NO: 64 - protein, 7 aa, Artificial Sequence
  Synthetic
AIMFPTR

SEQ ID NO: 65 - protein, 7 aa, Artificial Sequence
  Synthetic
ATPWVLP

SEQ ID NO: 66 - protein, 7 aa, Artificial Sequence
  VARIANT 5, 6, 7: Xaa = Any Amino Acid
  VARIANT 2: Xaa = Thr, Phe, or Val
  VARIANT 4: Xaa = Thr, Val, Phe, or Tyr
  Synthetic
AXPXXXX

SEQ ID NO: 67 - protein, 4 aa, Artificial Sequence
  Synthetic
ARPL

SEQ ID NO: 68 - protein, 4 aa, Artificial Sequence
  Synthetic
ARPI

SEQ ID NO: 69 - protein, 4 aa, Artificial Sequence
  Synthetic
ARPF

SEQ ID NO: 70 - protein, 4 aa, Artificial Sequence
  Synthetic
AKPL

SEQ ID NO: 71 - protein, 4 aa, Artificial Sequence
  Synthetic
AKPI

SEQ ID NO: 72 - protein, 4 aa, Artificial Sequence
  Synthetic
AKPF

SEQ ID NO: 73 - protein, 4 aa, Artificial Sequence
  Synthetic
AHPL

SEQ ID NO: 74 - protein, 4 aa, Artificial Sequence
  Synthetic
AHPI

SEQ ID NO: 75 - protein, 4 aa, Artificial Sequence
  Synthetic
AHPF

SEQ ID NO: 76 - protein, 4 aa, Artificial Sequence
  based on Drosophila melanogaster
AVPI

SEQ ID NO: 77 - protein, 4 aa, Artificial Sequence
  based on Drosophila melanogaster
ATPF

SEQ ID NO: 78 - protein, 4 aa, Artificial Sequence
  based on Drosophila melanogaster
AVPF

SEQ ID NO: 79 - protein, 4 aa, Artificial Sequence
  based on Drosophila melanogaster
AVPA

SEQ ID NO: 80 - protein, 4 aa, Artificial Sequence
  based on Drosophila melanogaster
AVPS

SEQ ID NO: 81 - protein, 29 aa, Artificial Sequence
  Homologous sequence
MPRFMDYWEGLNRQIKIWFQNERRMKWKK

SEQ ID NO: 82 - protein, 68 aa, Artificial Sequence
  VARIANT 1-3, 6, 9, 10, 13, 14, 16, 18-21, 24, 30, 32, 33, 35,37, 40, 42-44, 46, 47, 49-51, 53-57, 59, 61, 62, 64, 66: Xaa = Any Amino Acid
  VARIANT 5, 17, 28, 29, 45, 68: Xaa = Hyrophobic Amino Acid (i. e., Ala, Cys, Ile, Leu, Met, Phe, Pro, Trp, Tyr, or Val)
  VARIANT 7: Xaa = Serine or Threonine
  VARIANT 8, 67: Xaa = Phenylalanine or Tyrosine
  VARIANT 12: Xaa = Proline or is absent
  VARIANT 15: Xaa = any amino acid or is absent.
  VARIANT (52)...(52): Xaa = Aspartic Acid or Glutamic Acid
  VARIANT (60)...(60): Xaa = Basic Amino Acid (i. e., Arg, His, or Lys)
  Based on Homo sapiens
XXXRXXXXXXWXXXXXXXXXXLAXAGFXXXGXXDXVXCFXCXXXXXXWXXXXXXXXXHXXXXPXCXXX

SEQ ID NO: 83 - protein, 68 aa, Artificial Sequence
  VARIANT 1, 2, 3, 6, 9, 10, 13, 14, 16, 19, 24, 29, 30, 32, 33,37, 46, 47, 49-51, 56, 57, 59, 61, 62, 64, 66: Xaa = Any Amino Acid
  VARIANT 5, 36, 68: Xaa = Hydrophobic Amino Acid
  VARIANT 7: Xaa = Serine or Threonine
  VARIANT 12: Xaa = Proline or is Absent
  VARIANT 15: Xaa = Any Amino Acid or is Absent
  VARIANT 23: Xaa = Serine or Alanine
  VARIANT (52)...(52): Xaa = Aspartic Acid or Glutamic Acid
  VARIANT (60)...(60): Xaa = Any Basic Amino Acid
  VARIANT (67)...(67): Xaa = Phenylalanine or Tyrosine
  based on Homo sapiens
XXXRXXXFXXWXXXXXVNXENLXXAGFYXXGXXDKXXCFHCGGGLXXWXXXXDPWXXHXXXXPXCXXX

SEQ ID NO: 84 - protein, 9 aa, Artificial Sequence
  based on Drosophila melanogaster
AVPFYLPGG

SEQ ID NO: 85 - protein, 9 aa, Artificial Sequence
  based on Drosophila melanogaster
  BLOCKED 1: Ala at position 1 is Fmoc-Ala.
  MOD_RES 5, 8: Amino acids at positions 5 and 8 are butylated
AVPFYLPGG

(19) United States
(12) Patent Application Publication
(10) Pub. No.: US 2003/0157522 A1
(43) Pub. Date: Aug. 21, 2003

(54) Methods and reagents for peptide-BIR interaction screens

(76) Inventors: Alain Boudreault, Montreal (CA); Robert G. Korneluk, Ottawa (CA); Eric LaCasse, Ottawa (CA); Peter Liston, Ottawa (CA)

Correspondence Address: CLARK & ELBING LLP, 101 FEDERAL STREET, BOSTON, MA, 02110, US

(21) Appl. No.: 10/293,371

(22) Filed: Nov. 12, 2002

Related U.S. Application Data
(60) Provisional application No. 60/332,300, filed on Nov. 9, 2001.
(60) Provisional application No. 60/370,934, filed on Apr. 8, 2002.

(51) Int. Cl. C12Q 1/68; G01N 33/53; C07H 21/04; C12P 21/02; C12N 5/06; C07K 14/47

(52) U.S. Cl. 435/6; 435/7.1; 435/69.1; 435/320.1; 435/325; 530/324; 536/23.2

(57) ABSTRACT

The invention features a substantially pure polypeptide having a length of less than 100 amino acids and capable of forming a complex with a polypeptide that includes a BIR domain. The invention also features displacement assays in which the ability of a candidate compound to disrupt the interaction between a BIR domain-containing polypeptide and a polypeptide of the invention is indicative of the ability of the candidate compound to modulate IAP biological activity.

### CROSS-REFERENCE TO RELATED APPLICATIONS

This application claims benefit from copending U.S. Provisional Application Nos. 60/332,300 (filed Nov. 9, 2001) and 60/370,934 (filed Apr. 8, 2002), each of which is incorporated by reference.

### FIELD OF THE INVENTION

The invention relates to the detection of therapeutic compounds that modulate apoptosis.

BACKGROUND OF THE INVENTION

One way by which cells die is referred to as apoptosis, or programmed cell death. Apoptosis often occurs as a normal part of the development and maintenance of healthy tissues. This process involves a series of coordinated events, which may be triggered by development cues, viral infections, or prompted by irreparable DNA damage caused by UV irradiation. Cellular quality control pathways are also intricately linked to the apoptotic pathway, which may be activated upon inappropriate entry or progression in the cell cycle. DNA synthesis, protein synthesis or protein folding errors that occur on a scale in which the cell cannot make the appropriate repairs, set in motion signals leading to cell death.

The apoptosis pathway is now known to play a critical role in embryonic development, viral pathogenesis, cancer, autoimmune disorders, and neurodegenerative diseases, as well as other events. The failure of an apoptotic response has been implicated in the development of cancer, autoimmune disorders, such as systemic lupus erythematosis and multiple sclerosis, and in viral infections, including those associated with herpes virus, poxvirus, and adenovirus.

The inhibitors of apoptosis, or IAPs, are a family of proteins possessing one or more baculovirus IAP repeat (BIR) domains. The classical human IAPs, XIAP, HIAP1 (also referred to as cIAP2), and HIAP2 (cIAP1) all possess three BIR domains and carboxy terminal RING zinc finger. The third BIR domain of the IAPs (BIR3) binds and inhibits caspase-9, a key protease responsible for initiating the cascade in response to genotoxic damage and many other triggers. The second BIR domain of these IAPs inhibits the activity of caspases-3 and -7, two downstream or effector caspases that are common to all apoptotic pathways. A requirement for the BIR domains has also been demonstrated for the interactions of IAPs with tumor necrosis factor-associated factor (TRAFs)-1 and -2, and to TAB1. The IAPs thus function as a ‘constraint’ on the caspase cascade, preventing or limiting caspase activation. Because of this central mode of action, the IAPs are capable of suppressing cell death from a wide variety of triggers, including chemotherapeutic drugs and irradiation.

Progress in the cancer field has now led to a new paradigm in cancer biology wherein neoplasia is viewed as a failure to execute normal pathways of programmed cell death. Normal cells receive continuous feedback from their environment through various intracellular and extracellular factors, and “commit suicide” if removed from this context. Cancer cells gain the ability to ignore or bypass these commands and continue inappropriate proliferation. Cancer therapies, including radiation and many chemotherapies, have traditionally been viewed as causing overwhelming cellular injury. New evidence suggests that cancer therapies actually work by triggering apoptosis.

Overexpression of one or more of the IAPs has been documented in most established cancer cell lines, as well as in primary tumor biopsy samples. Chromosome amplification of the 11q21-q23 region, which encompasses both HIAP1 and HIAP2, has been observed in a variety of malignancies, including medulloblastomas, renal cell carcinomas, glioblastomas, and gastric carcinomas.

Some esophageal squamous cell carcinomas (ESCs) also display this amplification, and transcriptional profiling has identified HIAP2 as the sole target gene that is consistently overexpressed in these tumors. Translocation of HIAP1 has also been documented in the development of some mucosa-associated lymphatic tissue lymphomas.

The X-ray crystallographic structure of XIAP was previously solved, revealing a critical binding pocket and groove on the surface of each BIR domain. Two mammalian mitochondrial proteins (Smac and Omi/Htra2), and four Drosophila proteins (Reaper, HID, Grim, and Sickle) that interfere with IAP function by binding to these sites on the BIR domain have been identified. Each of these IAP inhibitors possesses a short amino-terminal peptide sequence that fits into this binding pocket and disrupts IAP-caspase interactions. In several respects therefore, the BIR interaction surface resembles a protease catalytic site; the deep pocket and surface groove provide a highly specific binding site, linear peptide sequences are recognized, and multiple ‘substrates’ with similar, but non-identical, sequences have been identified. Although the overall folding of individual BIR domains is believed to be generally conserved, there are alterations in the amino acid sequences that form the binding pocket and groove that suggest that binding affinities might vary between each of the BIR domains.

It is thus desirable to develop anti-cancer therapeutics capable of targeting and inhibiting IAP activity.

### SUMMARY OF THE INVENTION

We have performed phage display peptide library screening to identify candidate peptide ligands that bind to the individual BIR domains of XIAP and some of the other IAPs (HIAP1 BIR3, HIAP2 BIR3, TsIAP). From this screening, we have identified multiple novel peptides capable of binding an individual BIR domain of XIAP and/or other IAPs (HIAP1 BIR3, HIAP2 BIR3, etc.). We have also developed a displacement assay as a method to identify candidate compounds capable of competing with these or other peptide ligands and binding to particular BIR domains. These candidate compounds are useful, for example, as anti-cancer agents, and as lead compounds for the identification of other anti-cancer agents.

Accordingly, in one aspect the present invention features peptides and derivatives thereof that are capable of binding to an IAP, particularly human XIAP, HIAP-1, or HIAP-2, and specifically inhibiting or blocking the binding of that IAP to a caspase protein (e.g., caspase-9) in vitro or in vivo. The peptides of the present invention are: Ala-Lys-Pro-Leu-Ala-Leu-Thr (Seq ID NO: 3), Ala-His-Pro-Gly-Met-Pro-Gln (Seq ID NO: 4), Ala-Thr-Pro-Trp-Val-Asp-Gln (Seq ID NO: 5), Ala-Arg-Pro-Phe-Ala-Thr-Tyr (Seq ID NO: 6), Ala-His-Pro-Val-Met-Pro-Gln (Seq ID NO: 7), Glu-Met-Arg-Leu-Gly-Leu-Glu (Seq ID NO: 8), Ala-Val-Pro-Leu-Ser-Thr-Gln (Seq ID NO: 9), Leu-Ser-Gly-Ala-Asn-Ser-Thr (Seq ID NO: 10), Ala-Arg-Pro-Phe-Ser-Ser-Pro (Seq ID NO: 11), Ala-Arg-Pro-Leu-Ser-Asn-Ile (Seq ID NO: 12), Ala-Leu-Pro-Leu-Ser-His-Val (Seq ID NO: 13), Ala-Thr-Pro-Val-Phe-Asp-Leu (Seq ID NO: 14), Ala-Lys-Pro-Phe-Pro-Ser-Ala (Seq ID NO: 15), Ala-Thr-Pro-Ile-Trp-Leu-Pro (Seq ID NO: 16), Ala-Asn-Pro-Phe-Leu-Ser-Asp (Seq ID NO: 17), Ala-Met-Pro-Tyr-Ala-Pro-Gly (Seq ID NO: 18), Ala-Thr-Ser-Phe-His-Asp-Ala (Seq ID NO: 19), Ala-Leu-Pro-Leu-Thr-Gln-Val (Seq ID NO: 20), Thr-Gly-Ala-Ser-His-Ala-Pro (Seq ID NO: 21), Ala-Glu-Ile-Phe-Trp-Leu-Pro (Seq ID NO: 26), Ala-Ile-Pro-Ile-Ala-Thr-Ser (Seq ID NO: 27), Ala-Lys-Pro-Trp-Ser-Pro-Lys (Seq ID NO: 28), Ala-Asn-Pro-Ile-Pro-Arg-Ser (Seq ID NO: 29), Ala-Phe-Pro-Phe-Pro-Ser-Ala (Seq ID NO: 30), Glu-Val-Pro-Val-Arg-Thr-Ser (Seq ID NO: 31), Ala-Phe-Pro-Phe-Pro-Ser-Ala (Seq ID NO: 32), Leu-Ile-Pro-Ile-Ala-Thr-Ser (Seq ID NO: 33), Ala-Ser-Pro-Ile-Thr-Lys-Thr (Seq ID NO: 34), Ala-Ile-Pro-Ile-Ala-Thr-Ser (Seq ID NO: 35), Ala-Met-Pro-Tyr-Ala-Ser-Pro (Seq ID NO: 36), Ser-Ile-Lys-Trp-Trp-Thr-Pro (Seq ID NO: 37), Ala-Ile-Pro-Ile-Ala-Thr-Ser (Seq ID NO: 38), Ala-lle-Pro-Ile-Ala-Thr-Ser (Seq ID NO: 39), Ala-Phe-Pro-Phe-Pro-Ser-Ala (Seq ID NO: 40), Ala-Phe-Pro-Phe-Pro-Ser-Ala (Seq ID NO: 41), Ala-Phe-Pro-Phe-Pro-Ser-Ala (Seq ID NO: 42), Ala-Lys-Pro-Trp-His-Phe-Lysl (Seq ID NO: 43), Ala-Thr-Pro-Trp-Val-Leu-Pro (Seq ID No: 47), Ala-Val-Arg-Phe-Pro-Pro-Met (Seq ID No: 48), Ala-Ser-Arg-Ile-Gly-Thr-Thr (Seq ID No: 49), Ala-Met-Pro-Tyr-Leu-Gly-Gly (Seq ID No: 50), Ala-Ile-Pro-Ile-Ala-Thr-Ser (Seq ID No: 51), Ala-Phe-Pro-Val-Ser-His-Asn (Seq ID No: 52), Ser-Gin-Pro-Phe-Phe-Pro-Phe (Seq ID No: 53), Ala-Ser-Pro-Tyr-Thr-Ile-Pro (Seq ID No: 54), Ala-Asn-Pro-Ile-Pro-Arg-Ser (Seq ID No: 55), Ala-His-Pro-Tyr-Phe-Ala-Ala (Seq ID No: 56), Ala-Asn-Pro-Ile-Pro-Arg-Ser (Seq ID No: 57), Ala-Thr-Pro-Trp-Val-Leu-Pro (Seq ID No: 58), Ala-Met-Pro-Tyr-Leu-Gly-Gly (Seq ID No: 59), Ala-Thr-Pro-Phe-Met-Ala-His (Seq ID No: 61), Ala-Phe-Pro-Val-Ser-His-Asn (Seq ID No: 62), Ala-Asn-Pro-Ile-Pro-Arg-Ser (Seq ID No: 63), Ala-Ile-Met-Phe-Pro-Thr-Arg (Seq ID No: 64), and Ala-Thr-Pro-Trp-Val-Leu-Pro (Seq ID No: 65).

Many of the peptides of the invention satisfy one of four consensus sequences shown below.

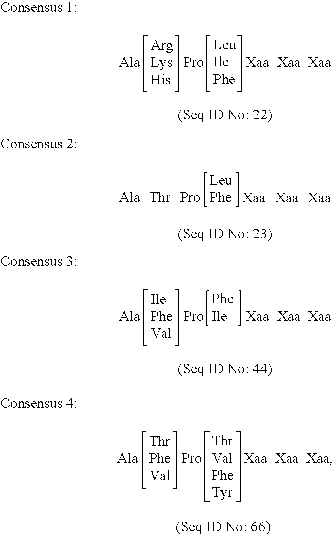

wherein Xaa is any amino acid or is absent. Accordingly, peptides satisfying any of the consensus sequences are also considered peptides of the invention. Preferred peptides include one of the following sequences: Ala-Arg-Pro-Leu (Seq ID No: 67), Ala-Arg-Pro-Ile (Seq ID No: 68), Ala-Arg-Pro-Phe (Seq ID No: 69), Ala-Lys-Pro-Leu (Seq ID No: 70), Ala-Lys-Pro-Ile (Seq ID No: 71), Ala-Lys-Pro-Phe (Seq ID No: 72), Ala-His-Pro-Leu (Seq ID No: 73), Ala-His-Pro-Ile (Seq ID No: 74), and Ala-His-Pro-Phe (Seq ID No: 75). Peptides of the invention are at least four amino acids in length (4mers) and desirably less than 100 amino acids, 50 amino acids, or even 20 amino acids. In particular embodiments, the peptides are 5mers, 6mers, 7mers, 8mers, 9mers, 10mers, 11mers, 12mers, 13mers, 14mers, 15mers, 16mers, 17mers, 18mers, 19mers, or 20mers.

In another aspect, the invention features a method for inhibiting or reducing the growth of a neoplastic cell, the method including the step of contacting the neoplastic cell with a cell growth-inhibiting amount of a recombinant protein that includes a peptide of the invention or a derivative thereof. In certain embodiments, the cell is a mammalian cell (e.g., a human cell). The contacting may be performed in vivo or ex vivo.

The invention also features a method for enhancing apoptosis, the method including the step of contacting a cell (e.g., a human cell) with an apoptosis-enhancing amount of a recombinant protein that includes a peptide of the invention or a derivative thereof. The contacting can be performed in vivo or ex vivo.

Pharmaceutical compositions that include a peptide of the invention or a derivative thereof and a pharmaceutically acceptable carrier or excipient are also part of the invention. The pharmaceutical compositions can be used, for example, for the treatment of neoplasms or enhancing apoptosis in a human or other mammal.

The invention also features nucleic acid molecule encoding a peptide of the invention. The nucleic acid molecule may be contained within an expression vector for expression in mammalian cells. In one example, the peptide is part of a ubiquitin fusion protein, which allows for expression of the peptide within a cell in the absence of a start methionine.

In a related aspect, the invention features a method for enhancing apoptosis by expressing in a cell (e.g., a human cell) a nucleic acid encoding a peptide of the invention. The contacting may be performed in vivo or ex vivo.

The invention also features a pharmaceutical composition that includes an expression vector encoding a peptide of the invention and a pharmaceutically acceptable carrier or excipient. The pharmaceutical compositions can be used, for example, for the treatment of neoplasms or enhancing apoptosis in a mammal.

In the another aspect, the invention features a method for identifying a compound that modulates IAP biological activity, this method includes the steps of: contacting a first polypeptide that includes a sequence selected from Seq ID NOs: 3-21, 26-43, 47-65, and 67-75, and a second polypeptide that includes a BIR domain to form a complex between the first polypeptide and second polypeptide; contacting this complex with a candidate compound; and measuring the displacement of the first polypeptide from the second polypeptide. Displacement of the first polypeptide from the second polypeptide is indicative that the candidate compound is a compound that modulates IAP biological activity. Measuring the displacement of the first polypeptide from the second polypeptide is relative to the amount of binding of the first and second polypeptide in the absence of a candidate compound.

The invention features another method for identifying a compound that modulates IAP biological activity, this method includes the steps of: contacting a first polypeptide that includes a sequence selected from Seq ID NOs: 3-21, 26-43, 47-65, and 67-75 and a second polypeptide that includes a BIR domain, the contacting being performed in the presence of a candidate compound; and measuring binding between the first and second polypeptides, wherein a reduction in the amount of binding, relative to the amount of binding between the first and said second polypeptides in the absence of candidate compound, indicates that the candidate compound is a compound that modulates IAP biological activity.

In either of the foregoing methods, the first polypeptide can consist essentially of a sequence selected from Seq ID NOs: 3-21,26-43, 47-65, and 67-75 or even consist of one of these sequences. The first polypeptide can also include a two amino acid residue sequence (e.g., Gly-Gly) linking the polypeptide to, e.g., a detectable label.

Either of the foregoing methods can further include the addition of a validation step which includes the steps of: providing neoplastic cells; contacting the neoplastic cells with the candidate compound; and measuring cell death in the neoplastic cells. An increase in cell death, relative to neoplastic cells not contacted with the candidate compound, indicates that the candidate compound is useful for the treatment of neoplastic disorders. This validation step can also include contacting the cells with a chemotherapeutic agent, for example, doxorubicin, daunorubicin, idarubicin, or mitoxantrone. In another validation step, the identified compound is further tested for binding with at least one, two, or three other polypeptides, each including a BIR domain having a sequence listed in FIG. 1A or 1B.

In either of these screening methods, the BIR domain of the second polypeptide is desirably one having a sequence listed in FIG. 1A. More desirably, the BIR domain is a BIR3 domain listed in FIG. 1B.

One or both of the polypeptides can be detectably labeled.

Various methods can be utilized to measure either binding of the first and second polypeptides or the displacement of the first polypeptide from the second polypeptide. For example, displacement of the polypeptides or binding of the polypeptides can be measured by employing the use of mass spectroscopy, surface plasmon resonance, fluorescence polarization, FRET, BRET, fluorescence quenching, ELISA, or RIA assays.

“Protein” or “polypeptide” or “peptide” means any chain of more than two natural or unnatural amino acids, regardless of post-translational modification (e.g., glycosylation or phosphorylation), constituting all or part of a naturally-occurring or non-naturally occurring polypeptide or peptide, as is described herein.

As used herein, a natural amino acid is a natural (α-amino acid having the L-configuration, such as those normally occurring in natural proteins. Unnatural amino acid refers to an amino acid, which normally does not occur in proteins, e.g., an epimer of a natural α-amino acid having the L configuration, that is to say an amino acid having the unnatural D-configuration; or a (D,L)-isomeric mixture thereof; or a homologue of such an amino acid, for example, a β-amino acid, an α,α-disubstituted amino acid, or an α-amino acid wherein the amino acid side chain has been shortened by one or two methylene groups or lengthened to up to 10 carbon atoms, such as an α-amino alkanoic acid with 5 up to and including 10 carbon atoms in a linear chain, an unsubstituted or substituted aromatic (α-aryl or α-aryl lower alkyl), for example, a substituted phenylalanine or phenylglycine.

As used herein, a “peptide of the invention” refers to a linear compound comprising the amino acid sequences and containing only natural amino acids which are linked by peptide bonds and which are in an unprotected form.

Specifically excluded from the peptides of the invention are peptides in which P1-P4 are Ala-Val-Pro-Ile (Seq ID No: 76), Ala-Thr-Pro-Phe (Seq ID No: 77), Ala-Val-Pro-Phe (Seq ID No: 78), Ala-Val-Pro-Ala (Seq ID No: 79), and Ala-Val-Pro-Ser (Seq ID No: 80).

The present invention also provides derivatives of the peptides of the invention. Such derivatives may be linear or circular, and include peptides having unnatural amino acids. Derivatives of the invention also include molecules wherein a peptide of the invention is non-covalently or preferably covalently modified by substitution, chemical, enzymatic or other appropriate means with another atom or moiety including another peptide or protein. The moiety may be “foreign” to a peptide of the invention as defined above in that it is an unnatural amino acid, or in that one or more natural amino acids are replaced with another natural or unnatural amino acid. Conjugates comprising a peptide or derivative of the invention covalently attached to another peptide or protein are also encompassed herein. Attachment of another moiety may involve a linker or spacer, e.g., an amino acid or peptidic linker. Derivatives of the invention also included peptides wherein one, some, or all potentially reactive groups, e.g., amino, carboxy, sulfhydryl, or hydroxyl groups are in a protected form.

The atom or moiety derivatizing a peptide of the invention may serve analytical purposes, e.g., facilitate detection of the peptide of the invention, favor preparation or purification of the peptide, or improve a property of the peptide that is relevant for the purposes of the present invention. Such properties include, cellular uptake, binding to an IAP, or suitability for in vivo administration, particularly solubility or stability against enzymatic degradation. Derivatives of the invention include a covalent or aggregative conjugate of a peptide of the invention with another chemical moiety, the derivative displaying essentially the same activity as the underivatized peptide of the invention, and a “peptidomimetic small molecule” which is modeled to resemble the three-dimensional structure of any of the amino acids of the invention. Examples of such mimetics are retro-inverso peptides (Chorev et al., Acc. Chem. Res. 26: 266-273, 1993). The designing of mimetics to a known pharmaceutically active compound is a known approach to the design of drugs based on a “lead” compound. This may be desirable, e.g., where the “original” active compound is difficult or expensive to synthesize, or where it is unsuitable for a particular mode of administration, e.g., peptides are considered unsuitable active agents for oral compositions as they tend to be quickly degraded by proteases in the alimentary canal.

Additional examples of derivatives within the above general definitions include the following:

(I) Cyclic peptides or derivatives including compounds with a disulfide bridge, a thioether bridge, or a lactam. Typically, cyclic derivatives containing a disulphide bond will contain two cysteines, which may be L-cysteine or D-cysteine. Advantageously, the N-terminal amino acid and the C-terminal amino acids are both cysteines. In such derivatives, as an alternative to cysteine, penicillamine (β,β-dimethyl-cysteine) can be used. Peptides containing thioether bridges are obtainable, e.g., from starting compounds having a free cysteine residue at one end and a bromo-containing building block at the other end (e.g., bromo-acetic acid). Cyclization can be carried out on solid phase by a selective deprotection of the side chain of cysteine. A cyclic lactam may be formed, e.g., between the γ-carboxy group of glutamic acid and the ε-amino group of lysine. As an alternative to glutamic acid, it is possible to use aspartic acid. As an alternative to lysine, ornithine or diaminobutyric acid may be employed. Also, it is possible to make a lactam between the side chain of aspartic acid or glutamic acid at the C-terminus and the α-amino group of the N-terminal amino acid. This approach is extendable to β-amino acids (e.g., β-alanine). Alternatively, glutamine residues at the N-terminus or C-terminus can be tethered with an alkenedyl chain between the side chain nitrogen atoms (Phelan et al., J. Amer. Chem. Soc. 119:455-460, 1997).

(II) Peptides of the invention, which are modified by substitution. In one example, one or more, preferably one or two, amino acids are replaced with another natural or unnatural amino acid, e.g., with the respective D-analog, or a mimetic. For example, in a peptide containing Phe or Tyr, Phe or Tyr may be replaced with another building block, e.g., another proteinogenic amino acid, or a structurally related analogue. Particular modifications are such that the conformation in the peptide is maintained. For example, an amino acid may be replaced by a α,α-disubstituted amino acid residue (e.g., α-aminoisobutyric acid, 1-amino-cyclopropane-1-carboxylic acid, 1-amino-cyclopentane-1-carboxylic acid, 1-amino-cyclohexane-1-carboxylic acid, 4-amino piperidine-4-carboxylic acid, and 1-amino-cycloheptane-1-carboxylic acid).

(III) Peptides of the invention detectably labeled with an enzyme, a fluorescent marker, a chemiluminescent marker, a metal chelate, paramagnetic particles, biotin, or the like. In such derivatives, the peptide of the invention is bound to the conjugation partner directly or by way of a spacer or linker group, e.g., a (peptidic) hydrophilic spacer. Advantageously, the peptide is attached at the N- or C-terminal amino acid. For example, biotin may be attached to the N-terminus of a peptide of the invention via a serine residue or the tetramer Ser-Gly-Ser-Gly.

(IV) Peptides of the invention carrying one or more protecting groups at a potentially reactive side group, such as amino-protecting group, e.g., acetyl, or a carboxy-protecting group. For example, the C-terminal carboxy group of a compound of the invention may be present in form of a carboxamide function. Suitable protecting groups are commonly known in the art. Such groups may be introduced, for example, to enhance the stability of the compound against proteolytic degradation.

By a “derivative” of a peptide of the invention is also meant a compound that contains modifications of the peptides or additional chemical moieties not normally a part of the peptide. Modifications may be introduced into the molecule by reacting targeted amino acid residues of the peptide with an organic derivatizing agent that is capable of reacting with selected side chains or terminal residues. Methods of derivatizing are described below.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl) propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are generally derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate; pyridoxal phosphate; pyridoxal; chloroborohydride; trinitrobenzenesulfonic acid; O-methylissurea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pK^{a} of the guanidine functional group. Furthermore, these reagents may react with the groups of lysine as well as the arginine epsilon-amino group.

Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3 (4 azonia 4,4-dimethylpentyl) carbodiimide. Aspartyl and glutamyl residues can also be converted to asparaginyl and glutaminyl residues by reaction with ammonium ions.

Glutaminyl and asparaginyl residues are frequently deamidated to the corresponding glutamyl and aspartyl residues. Alternatively, these residues are deamidated under mildly acidic conditions. Either form of these residues falls within the scope of this invention.

Peptides of the invention or derivatives thereof may be fused or attached to another protein or peptide, e.g., a protein or peptide serving as internalization vector, such as another peptide facilitating cellular uptake, e.g., a “penetratin.” An exemplary penetratin-containing derivative according to the invention is, e.g., a peptide comprising the sixteen amino acid sequence from the homeodomain of the Antennapedia protein (Derossi et al., J. Biol. Chem. 269:10444-10450, 1994), particularly a peptide having the amino acid sequence: Met-Pro-Arg-Phe-Met-Asp-Tyr-Trp-Glu-Gly-Leu-Asn-Arg-Gln-Ile-Lys-Ile-Trp-Phe-Gln-Asn-Glu-Arg-Arg-Met-Lys-Trp-Lys-Lys (Seq ID No: 81), or including a peptide sequence disclosed by Lin et al. (J. Biol. Chem. 270:14255-14258, 1995).

Polypeptides or derivatives thereof may be fused or attached to another protein or peptide, e.g., as a glutathione-S-transferase (GST) fusion polypeptide. Other commonly employed fusion polypeptides include, but are not limited to, maltose-binding protein, Staphylococcus aureus protein A, polyhistidine, and cellulose-binding protein.

By a “peptidomimetic small molecule” of a peptide is meant a small molecule that exhibits substantially the same BIR-binding properties as the peptide itself.

By “candidate compound” is meant a chemical, be it naturally-occurring or artificially-derived, that is assayed for its ability to modulate a polypeptide-peptide or protein-protein interaction, by employing one of the assay methods described herein. Test compounds may include, for example, peptides, polypeptides, synthesized organic molecules, naturally occurring organic molecules, nucleic acid molecules, and components thereof.

By “assaying” is meant analyzing the effect of a treatment, be it chemical or physical, administered to whole animals or cells derived there from. The material being analyzed may be an animal, a cell, a lysate or extract derived from a cell, or a molecule derived from a cell. The analysis may be, for example, for the purpose of detecting altered protein function, protein stability, altered protein-protein interactions, altered protein-peptide interactions, altered protein biological activity. The means for analyzing may include, for example, enzymatic assays, binding assays, immunoprecipitation, phosphorylation assays, and methods known to those skilled in the art for detecting nucleic acids and polypeptides.

By “cancer” or “neoplasia” is meant a cell or tissue multiplying or growing in an abnormal manner. Cancer growth is uncontrolled and progressive, and occurs under conditions that would not elicit, or would cause cessation of, multiplication of normal cells.

“Apoptosis” means the process of cell death wherein a dying cell displays a set of well-characterized biochemical hallmarks that include cell membrane blebbing, cell soma shrinkage, chromatin condensation, and DNA laddering. Cells that die by apoptosis include neurons (e.g., during the course of neurodegenerative diseases such as stroke, Parkinson's disease, and Alzheimer's disease), cardiomyocytes (e.g., after myocardial infarction or over the course of congestive heart failure), and cancer cells (e.g., after exposure to radiation or chemotherapeutic agents). Environmental stress (e.g., hypoxic stress) that is not alleviated may cause a cell to enter the early phase of the apoptotic pathway, which is reversible (i.e., cells at the early stage of the apoptotic pathway can be rescued). At a later phase of apoptosis (the commitment phase), cells cannot be rescued, and, as a result, are committed to die.

By “enhancing apoptosis” is meant increasing the number of cells that apoptose in a given cell population. Preferably the cell population is selected from a group including ovarian cancer cells, breast cancer cells, pancreatic cancer cells, T cells, neuronal cells, fibroblasts, or any other cell line known to proliferate in a laboratory setting. It will be appreciated that the degree of apoptosis enhancement provided by an apoptosis-enhancing compound in a given assay will vary, but that one skilled in the art can determine the statistically significant change in the level of apoptosis that identifies a compound that enhances apoptosis otherwise limited by an IAP. Desirably “enhancing apoptosis” means that the increase in the number of cells undergoing apoptosis is at least 25%, more preferably the increase is 50%, and most preferably the increase is at least one-fold. Desirably the sample monitored is a sample of cells that normally undergo insufficient apoptosis (i.e., cancer cells). Methods for detecting changes in the level of apoptosis (i.e., enhancement or reduction) are described herein.

By “IAP gene” is meant a gene encoding a polypeptide having at least one BIR domain that is capable of modulating (inhibiting or enhancing) apoptosis in a cell or tissue when provided by other intracellular or extracellular delivery methods (see, e.g., U.S. Pat. No. 5,919,912, U.S. Ser. No. 08/576,965, U.S. Pat. No. 6,107,041, and U.S. Pat. No. 6,300,492, each of which is hereby incorporated by reference).

By “IAP” is meant a polypeptide, or fragment thereof, encoded by an IAP gene. Exemplary IAPs are XIAP, HIAP1, HIAP2, NAIP, and testis-specific IAP.

By “IAP biological activity” is meant the regulation of apoptosis through the interaction of IAP gene products with pro- and anti-apoptotic proteins. For example, IAP biological activity is at least in part directed to regulating apoptosis. This is facilitated through interactions of IAP molecules with caspases, wherein this interaction is associated with the inhibition of apoptosis. Displacement of IAPs from caspases may lead to the release of the inhibitory effects of IAPs. Certain binding proteins have been demonstrated to be associated with IAPs. Specifically, the BIR3 domain may interact with caspase 9, and polypeptides with an exposed AxPx peptide sequence. One mechanism by which the BIR3 domain regulates apoptotic function is through the binding of this region to caspase 9, in an AxPx-dependent manner. Soluble AxPx sequences displace caspase 9 from BIR3, resulting in caspase 9 proteolytic activity, an initiator of apoptosis, if left unabated.

By “BIR domain” is meant a domain having the amino acid sequence of the consensus sequence: Xaa1-Xaa1-Xaa1-Arg-Xaa3-Xaa1-Xaa4-Xaa5-Xaa1-Xaa1-Trp-Xaa6-Xaa1-Xaa1-Xaa2-Xaa1-Xaa3-Xaa1-Xaa1-Xaa1-Xaa1-Leu-Ala-Xaa1-Ala-Gly-Phe-Xaa3-Xaa3-Xaa1-Gly-Xaa1-Xaa1-Asp-Xaa1-Val-Xaa1-Cys-Phe-Xaa1-Cys-Xaa1-Xaa1-Xaa1-Xaa3-Xaa1-Xaa1-Trp-Xaa1-Xaa1-Xaa1-Xaa7-Xaa1-Xaa1-Xaa1-Xaa1-Xaa1-His-Xaa1-Xaa8-Xaa1-Xaa1-Pro-Xaa1-Cys-Xaa1-Xaa5-Xaa3 (Seq ID NO: 82), wherein Xaa1 is any amino acid, Xaa2 is any amino acid or is absent, Xaa3 is a hydrophobic amino acid (i.e., Ala, Cys, Ile, Leu, Met, Phe, Pro, Trp, Tyr, or Val), Xaa4 is serine or threonine, Xaa5 is phenylalanine or tyrosine, Xaa6 is proline or is absent, Xaa7 is aspartic or glutamic acid, Xaa8 is a basic amino acid (i.e., Arg, His, or Lys), Xaa9 is serine or alanine. Desirably the sequence is substantially identical to one of the BIR domain sequences provided for human or mouse XIAP, HIAP1, or HIAP2.

By “BIR3 domain” is meant a domain having the amino acid sequence of the consensus sequence: Xaa1-Xaa1-Xaa1-Arg-Xaa3-Xaa1-Xaa4-Phe-Xaa1-Xaa1-Trp-Xaa6-Xaa1-Xaa1-Xaa2-Xaa1-Val-Asn-Xaa1-Glu-Asn-Leu-Xaa9-Xaa1-Ala-Gly-Phe-Tyr-Xaa1-Xaa1-Gly-Xaa1-Xaa1-Asp-Lys-Xaa3-Xaa1-Cys-Phe-His-Cys-Gly-Gly-Gly-Leu-Xaa1-Xaa1-Trp-Xaa1-Xaa1-Xaa1-Xaa7-Asp-Pro-Trp-Xaa1-Xaa1-His-Xaa1-Xaa8-Xaa1-Xaa1-Pro-Xaa1-Cys-Xaa1-Xaa5-Xaa3 (Seq ID NO: 83), wherein Xaa1 is any amino acid, Xaa2 is any amino acid or is absent, Xaa3 is a hydrophobic amino acid, Xaa4 is serine or threonine, Xaa5 is phenylalanine or tyrosine, Xaa6 is proline or is absent, Xaa7 is aspartic or glutamic acid, Xaa8 is any basic amino acid, Xaa9 is serine or alanine. Desirably the sequence is substantially identical to one of the BIR3 domain sequences provided herein for human or mouse XIAP, HIAP1, HIAP2, TsIAP, or Livin/KIAP.

By “substantially identical” is meant a polypeptide or nucleic acid exhibiting at least 85%, but preferably 90%, more preferably 95%, most preferably 97%, or even 99% identity to a reference amino acid or nucleic acid sequence.

Sequence identity is typically measured using a sequence analysis program (e.g., BLAST 2; Tatusova et al., FEMS Microbiol Lett. 174:247-250, 1999) with the default parameters specified therein. Conservative substitutions typically include substitutions within the following groups: glycine, alanine, valine, isoleucine, leucine; aspartic acid, glutamic acid, asparagine, glutamine; serine, threonine; lysine, arginine; and phenylalanine and tyrosine.

By “modulating” is meant conferring a change, either by decrease or increase, in IAP biological activity that is naturally present within a particular cell or sample. Preferably, the change in response is at least 5%, more preferably, the change in response is 20% and most preferably, the change in response level is a change of more than 50% relative to the levels observed in naturally occurring IAP biological activity.

By “substantially pure polypeptide” is meant a polypeptide or peptide that has been separated from the components that naturally accompany it. Typically, the polypeptide is substantially pure when it is at least 60%, by weight, free from the proteins and naturally-occurring organic molecules with which it is naturally associated. Preferably the polypeptide is an IAP polypeptide that is at least 75%, more preferably at least 90%, and most preferably at least 99%, by weight, pure. A substantially pure IAP polypeptide may be obtained, for example, by extraction from a natural source (e.g., a fibroblast, neuronal cell, or lymphocyte) by expression of a recombinant nucleic acid encoding an IAP polypeptide, or by chemically synthesizing the polypeptide. Purity can be measured by any appropriate method, e.g., by column chromatography, polyacrylamide gel electrophoresis, or HPLC analysis.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides include those derived from eukaryotic organisms but synthesized in E. Coli or other prokaryotes.

By “substantially pure DNA” is meant DNA that is free of the genes that, in the naturally-occurring genome of an organism from which the DNA of the invention might be derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By “positioned for expression” is meant that the nucleic acid is positioned adjacent to a DNA sequence that directs transcription and translation of the nucleic acid (i.e., facilitates the production of, e.g., an IAP-interacting peptide).

By “promoter” is meant a minimal sequence sufficient to direct transcription. Also included in the invention are those promoter elements that are sufficient to render allow for cell type-specific, tissue-specific, or that are inducible by external signals or agents; such elements may be located in the 5′ or 3′regions of the native gene.

By “operably linked” is meant that a gene and one or more regulatory sequences are connected in such a way as to permit gene expression when the appropriate molecules (e.g., transcriptional activator proteins) are bound to the regulatory sequences.

By “detectably-labeled” is meant any means for marking and identifying the presence of a molecule, e.g., a BIR domain-interacting peptide, a BIR domain polypeptide, a nucleic acid encoding the same, or a peptidomimetic small molecule. Methods for detectably-labeling a molecule are well known in the art and include, without limitation, radioactive labeling (e.g., with an isotope such as ³²p or ³⁵S) and nonradioactive labeling (e.g., chemiluminescent labeling or fluorescein labeling).

If an analysis involves identifying more than one distinct molecule, the molecules can be differentially labeled using markers, which can distinguish the presence of multiply distinct molecules. For example, a BIR domain-interacting peptide can be labeled with fluorescein and a BIR domain polypeptide can be labeled with Texas Red. The presence of the BIR domain-interacting peptide can be monitored simultaneously with the presence of the BIR domain.

Other features and advantages of the invention will be apparent from the following description of the preferred embodiments thereof, and from the claims.

### BRIEF DESCRIPTION OF THE DRAWINGS

FIGS. 1A and 1B are multi-sequence alignments of BIR domains from selected IAP polypeptide sequences. FIG. 1A shows sequence alignments of BIR domains from selected human IAP genes, whereas FIG. 1B illustrates alignments of multi-species BIR3 domains.

FIGS. 2A and 2B are schematic illustrations representing the basic configuration of the high throughput biochemical assay, which employs fluorescence-polarization detection of changes in ligand rotation in solution due to binding to an acceptor.

FIGS. 3A-3C are graphs depicting the fluorescence polarization assay described in FIGS. 2A and 2B, using HID and XIAP BIR3 as ligand and acceptor, respectively. FIG. 3A shows BIR3 specificity to a HID peptide. FIG. 3B shows cold HID polypeptide can compete with labeled HID polypeptide for XIAP BIR3 interaction. FIG. 3C shows that several peptides are able to bind to BIR3 and displace the HID peptide.

FIGS. 4A and 4B are schematic illustrations showing a representative scattergram of a dataset and the Qc of materials used in the HID/XIAP BIR3 high throughput screening assay of FIG. 2B. Data is analyzed using Spotfire data analysis software.

FIG. 5 is a scattergram illustration summarizing the results of a dataset obtained following the HID/XIAP BIR3 high throughput screening assay of FIG. 2B. Primary screening raw data has been transformed as a percentage of binding activity.

FIG. 6 is bar graph illustration revalidating positive hits in the HID/XIAP BIR3 high throughput screening assay of FIG. 2B.

FIG. 7 is a table illustrating selectivity of hits screened in HID/XIAP BIR3 high throughput screening assay of FIG. 2B.

FIG. 8 is a schematic illustration showing the effect of a compound identified as being capable of dislodging a HID 7mer from XIAP BIR3 on cell survival of T24 bladder carcinoma cells after 24 hours of exposure to 10 μg/ml adriamycin.

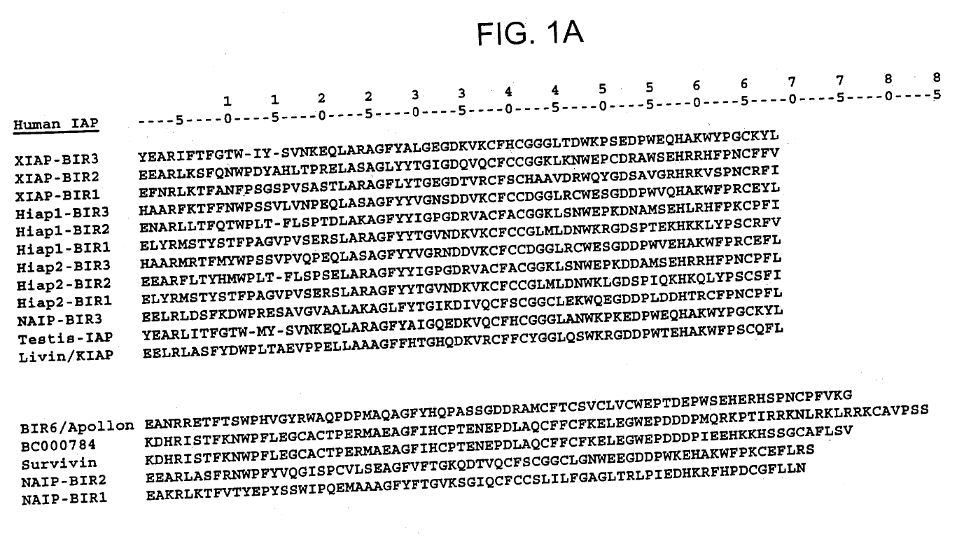

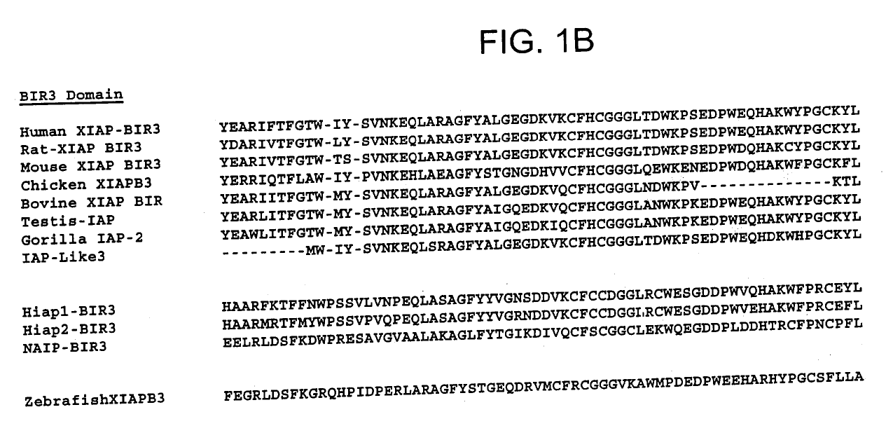

### DETAILED DESCRIPTION

We have discovered a method and reagents for screening for compounds capable of binding to IAPs and preventing their interaction with caspases. Compounds identified by this method, as well as derivatives thereof, are useful, for example, as therapeutic agents for the treatment of cancer and other neoplasms. Identification of peptides capable of binding IAP BIR domains We undertook the following approach to identifying peptides capable of specifically binding to BIR domains of various IAPs. A specific BIR domain was first expressed as a GST fusion, and then incubated with a phage display library, which allowed for the expression of random hexamers without the requirement for an N-terminal methionine residue. Phage were eluted, amplified, and re-screened for a total of three rounds. This approach led to the identification of novel IAP-binding peptide sequences having an AxPx-type motif. One consensus peptide sequence (Seq ID No: 22; Table 1) could be distinguished from previously produced peptides by the presence of a basic residue (Arg, His, or Lys) in the second position (P2). This was only found for peptides that bind BIR3 of XIAP, and not to other BIRs of XIAP or other IAPs tested.

The results of the peptide library screening are as follows:

Table 1 depicts the sequences from 20 phages from the third round of selection with GST-XIAP BIR3. One class of peptides (Seq ID No: 23) has a hydrophobic amino acid in P2, similar to the sequence of Smac and caspase-9. The other class contains a positively charged amino acid in P2. No clear consensus emerges for P5, P6, or P7.

TABLE 1

Phage display results with XIAP BIR3

| | | | | | | | |
|---|---|---|---|---|---|---|---|
| P1 | P2 | P3 | P4 | P5 | P6 | P7 | Seq ID No. |

| | | | | | | | | |
|---|---|---|---|---|---|---|---|---|
| Smac: | Ala | Val | Pro | Ile | Ala | Gln | Lys | 1 |
| Caspase-9: | Ala | Thr | Pro | Phe | Gln | Glu | Gly | 2 |
| XIAP BIR 3 | Ala | Lys | Pro | Leu | Ala | Leu | Thr | 3 |
| binding | Ala | His | Pro | Gly | Met | Pro | Gln | 4 |
| peptides: | Ala | Thr | Pro | Trp | Val | Asp | Gln | 5 |
| | Ala | Arg | Pro | Phe | Ala | Thr | Tyr | 6 |
| | Ala | His | Pro | Val | Met | Pro | Gln | 7 |
| | Glu | Met | Arg | Leu | Gly | Leu | Glu | 8 |
| | Ala | Val | Pro | Leu | Ser | Thr | Gln | 9 |
| | Leu | Ser | Gly | Ala | Asn | Ser | Thr | 10 |
| | Ala | Arg | Pro | Phe | Ser | Ser | Pro | 11 |
| | Ala | Arg | Pro | Leu | Ser | Asn | Ile | 12 |
| | Ala | Leu | Pro | Leu | Ser | His | Val | 13 |
| | Ala | Thr | Pro | Val | Phe | Asp | Leu | 14 |
| | Ala | Lys | Pro | Phe | Pro | Ser | Ala | 15 |
| | Ala | Thr | Pro | Ile | Trp | Leu | Pro | 16 |
| | Ala | Asn | Pro | Phe | Leu | Ser | Asp | 17 |
| | Ala | Met | Pro | Tyr | Ala | Pro | Gly | 18 |
| | Ala | Thr | Ser | Phe | His | Asp | Ala | 19 |
| | Ala | Leu | Pro | Leu | Thr | Gln | Val | 20 |
| | Thr | Gly | Ala | Ser | His | Ala | Pro | 21 |
| Consensus: | | Arg | | Leu | | | | |
| | Ala | Lys | Pro | Ile | Xaa | Xaa | Xaa | 22 |
| | | His | | Phe | | | | |
| | Ala | Thr | Pro | Phe | Xaa | Xaa | Xaa | 23 |

Table 2 depicts sequences of phages from the third round of selection with GST-HIAP1 BIR3. Most peptides conformed to the pattern of Smac and caspase-9, with a hydrophobic amino acid at P2 and P4.

TABLE 2

Phage display results with HIAP1 BIR3

| | | | | | | | |
|---|---|---|---|---|---|---|---|
| P1 | P2 | P3 | P4 | P5 | P6 | P7 | Seq ID No. |

| | | | | | | | | |
|---|---|---|---|---|---|---|---|---|
| Smac: | Ala | Val | Pro | Ile | Ala | Gln | Lys | 24 |
| Caspase-9: | Ala | Thr | Pro | Phe | Gln | Glu | Gly | 25 |
| HIAP1 BIR3 | Ala | Glu | Ile | Phe | Trp | Leu | Pro | 26 |
| binding | Ala | Ile | Pro | Ile | Ala | Thr | Ser | 27 |
| peptides: | Ala | Lys | Pro | Trp | Ser | Pro | Lys | 28 |
| | Ala | Asn | Pro | Ile | Pro | Arg | Ser | 29 |
| | Ala | Phe | Pro | Phe | Pro | Ser | Ala | 30 |
| | Glu | Val | Pro | Val | Arg | Thr | Ser | 31 |
| | Ala | Phe | Pro | Phe | Pro | Ser | Ala | 32 |
| | Leu | Ile | Pro | Ile | Ala | Thr | Ser | 33 |
| | Ala | Ser | Pro | Ile | Thr | Lys | Thr | 34 |
| | Ala | Ile | Pro | Ile | Ala | Thr | Ser | 35 |
| | Ala | Met | Pro | Tyr | Ala | Ser | Pro | 36 |
| | Ser | Ile | Lys | Trp | Trp | Thr | Pro | 37 |
| | Ala | Ile | Pro | Ile | Ala | Thr | Ser | 38 |
| | Ala | Ile | Pro | Ile | Ala | Thr | Ser | 39 |
| | Ala | Phe | Pro | Phe | Pro | Ser | Ala | 40 |
| | Ala | Phe | Pro | Phe | Pro | Ser | Ala | 41 |
| | Ala | Phe | Pro | Phe | Pro | Ser | Ala | 42 |
| | Ala | Lys | Pro | Trp | His | Phe | Lysl | 43 |
| Consensus: | | Ile | | Ile | | | | |
| | Ala | Phe | Pro | Phe | Xaa | Xaa | Xaa | 44 |
| | | Val | | Phe | | | | |

Table 3 depicts the sequences of phages from the third round of selection with GST-HIAP2 BIR3. Most peptides conformed to the pattern in Smac and caspase-9, with a hydrophobic amino acid in positions 2 and 4. Tyrosine in position 4 is observed more frequently in peptides that bind HIAP2 BIR3 than in peptides that bind XIAP or HIAP1 BIR3.

TABLE 3

Phage display results with HIAP2 BIR3

| | | | | | | | |
|---|---|---|---|---|---|---|---|
| P1 | P2 | P3 | P4 | P5 | P6 | P7 | Seq ID No. |

| | | | | | | | | |
|---|---|---|---|---|---|---|---|---|
| Smac: | Ala | Val | Pro | Ile | Ala | Gln | Lys | 45 |
| Caspase-9: | Ala | Thr | Pro | Phe | Gln | Glu | Gly | 46 |
| HAIP2 BIR 3 | Ala | Thr | Pro | Trp | Val | Leu | Pro | 47 |
| binding | Ala | Val | Arg | Phe | Pro | Pro | Met | 48 |
| peptides: | Ala | Ser | Arg | Ile | Gly | Thr | Thr | 49 |
| | Ala | Met | Pro | Tyr | Leu | Gly | Gly | 50 |
| | Ala | Ile | Pro | Ile | Ala | Thr | Ser | 51 |
| | Ala | Phe | Pro | Val | Ser | His | Asn | 52 |
| | Ser | Gln | Pro | Phe | Phe | Pro | Phe | 53 |
| | Ala | Ser | Pro | Tyr | Thr | Ile | Pro | 54 |
| | Ala | Asn | Pro | Ile | Pro | Arg | Ser | 55 |
| | Ala | His | Pro | Tyr | Phe | Ala | Ala | 56 |
| | Ala | Asn | Pro | Ile | Pro | Arg | Ser | 57 |
| | Ala | Thr | Pro | Trp | Val | Leu | Pro | 58 |
| | Ala | Met | Pro | Tyr | Leu | Gly | Gly | 59 |
| | Ala | Thr | Pro | Phe | Met | Ala | His | 61 |
| | Ala | Phe | Pro | Val | Ser | His | Asn | 62 |
| | Ala | Asn | Pro | Ile | Pro | Arg | Ser | 63 |
| | Ala | Ile | Met | Phe | Pro | Thr | Arg | 64 |
| | Ala | Thr | Pro | Trp | Val | Leu | Pro | 65 |
| Consensus: | | | | Thr | | | | |
| | | Thr | | Val | | | | |
| | Ala | Phe | Pro | Phe | Xaa | Xaa | Xaa | 66 |
| | | Val | | Phe | | | | |
| | | | | Tyr | | | | |

As is described below, peptides of the invention, or derivatives thereof, can be used for as therapeutic agents for the treatment of cancer and other neoplasms, and for generally enhancing apoptosis. Below we describe one particular method for delivering the peptides to a cell.

Producing the peptides of the invention, or derivatives thereof, as part of a ubiquitin fusion allows for the expression of the peptides in the cytosol without an N-terminal methionine. This method also eliminates the requirement for co-expression of a protease and the triggering of apoptosis and activation of caspases to cleave AxPx-caspase substrate fusions or to release mitochondrially targeted proteins which have their AxPx motifs revealed by cleavage of their leader peptide or by other proteolytic cleavages induced by proteins such as HtrA2/Omi. Each of these three latter approaches may pose problems to the accurate assessment of the cell death-inducing properties of the AxPx-containing molecules, as well as their IAP antagonistic effects. Eukaryotic protein translation requires a methionine as the start codon. Therefore, the expression of methionine-containing peptides and polypeptides may not yield IAP antagonistic molecules unless the methionine is removed. One cannot rely on methionine aminopeptidases to remove the N-terminal methionine from a polypeptide chain, as the resultant polypeptide may still be inactive due to acetylation of the N-terminus or the removal of additional amino-terminal residues. Another approach involves co-expression of a protease to generate a mature polypeptide chain with an AxPx motif downstream of a known specific protease sites (e.g., factor Xa, thrombin, HIV protease, caspases). However, the co-expression of a protease may result in cell death due to the multitude of cellular targets for the protease—some essential for cell survival—that will be incidentally cleaved by the protease.

The use of ubiquitin-fusion proteins for expressing peptides in the cell cytosol is described in U.S. Pat. Nos. 6,294,330; 6,287,858; 6,281,329; 6,180,343; 6,068,994; 5,914,254; 5,879,905; and 5,847,097, each of which is hereby incorporated by reference.

Modulation of Apoptosis with BIR-Interacting Peptides or Proteins Including Domains Corresponding to BIR-Interacting Peptides

The BIR-interacting peptides of the invention may themselves be administered to a cell that is expected to require enhanced apoptosis. The BIR-interacting peptide may be produced and isolated by any one of many standard techniques. Administration of such a peptide to neoplastic cells can be carried out by any of the methods for direct protein administration, as described herein.

If desirable, derivatization with bifunctional agents can be used for cross-linking the peptide to a macromolecular carrier. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophanyl)dithio]propioimidate yield photoactivatable intermediates that are capable of forming crosslinks in the presence of light.

Other modifications include hydroxylation of proline and lysine, phosphorylation of hydroxyl groups of seryl or theonyl residues, methylation of the α-amino groups of lysine, arginine, and histidine side chains (T. E. Creighton, Proteins: Structure and Molecule Properties, W. H. Freeman & Co., San Francisco, pp. 79-86 (1983)), acetylation of the N-terminal amine, and amidation of the C-terminal carboxyl groups.

Such derivatized moieties may improve the peptide's solubility, absorption, biological half life, and the like. The moieties may alternatively eliminate or attenuate any undesirable side effect of the peptide. Moieties capable of mediating such effects are disclosed, for example, in Remington: The Science and Practice of Pharmacy (20th ed.), ed. A. R. Gennaro, 2000, Lippincott Williams & Wilkins, Philadelphia, Pa. and the Encyclopedia of Pharmaceutical Technology, eds. J. Swarbrick and J. C. Boylan, 1988-1999, Marcel Dekker, New York, N.Y.

A derivative according to the invention may involve one or multiple modifications as compared to a peptide of the invention, e.g. carry one or more of the above-defined modifications. In other words, a derivative of the invention is intended to include compounds derivable from or based on a peptide of the invention or another derivative of the invention. The preferred derivatives of the invention are capable of binding to an IAP BIR domain (e.g., XIAP BIR3, HIAP1 BIR3, or HIAP2 BIR3) and selectively inhibiting or blocking the binding of the IAP to its natural caspase partner(s) (e.g., the binding of XIAP to caspase-9).

The peptides and derivatives of the present invention can be readily prepared according to well-established, standard liquid or solid-phase peptide synthesis methods, general descriptions of which are broadly available, or they may be prepared in solution, by the liquid phase method or by any combination of solid-phase, liquid phase and solution chemistry, e.g., by first completing the respective peptide portion and then, if desired and appropriate, after removal of any protecting groups being present.

Identification of Small-Molecule Disrupters of Binding Between an IAP and a BIR Interacting Peptide

As is described below, peptides or derivatives thereof, can be employed in screens for drug discovery binding assays. Such screens would identify peptidomimetic small molecules capable of displacing natural IAP-caspase interactions within the cell. Below we describe one particular method for screening of such compounds.

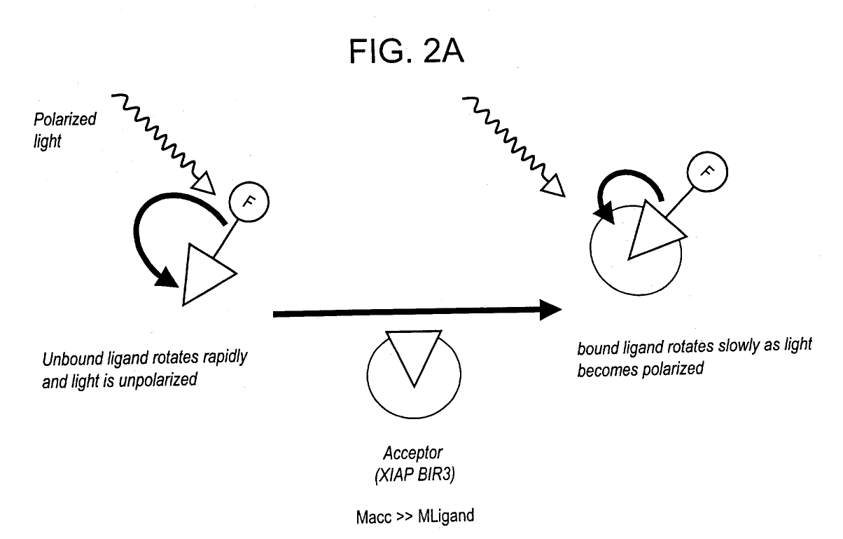

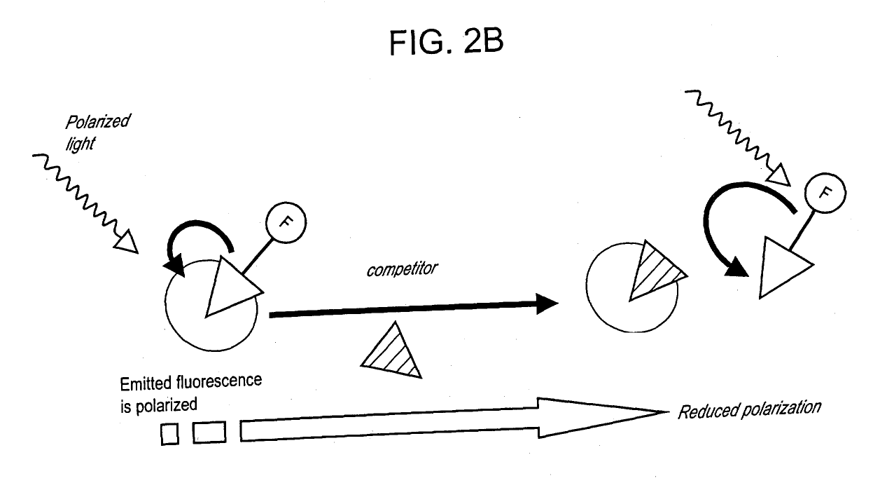

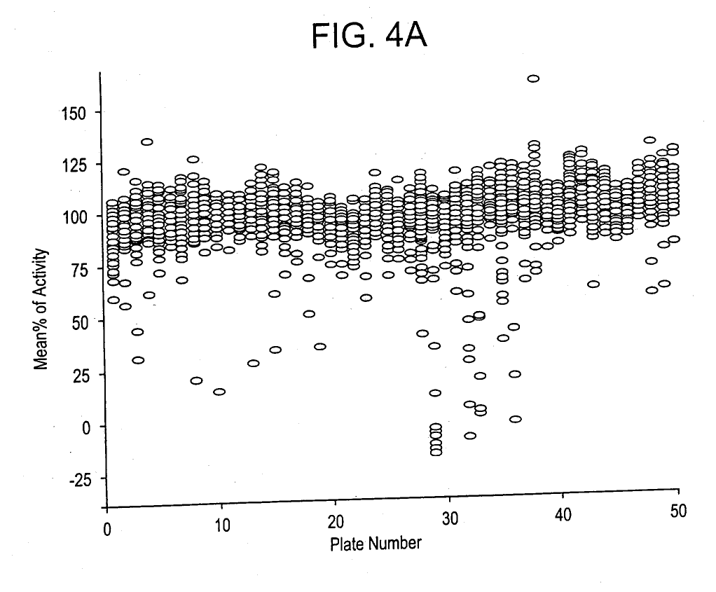

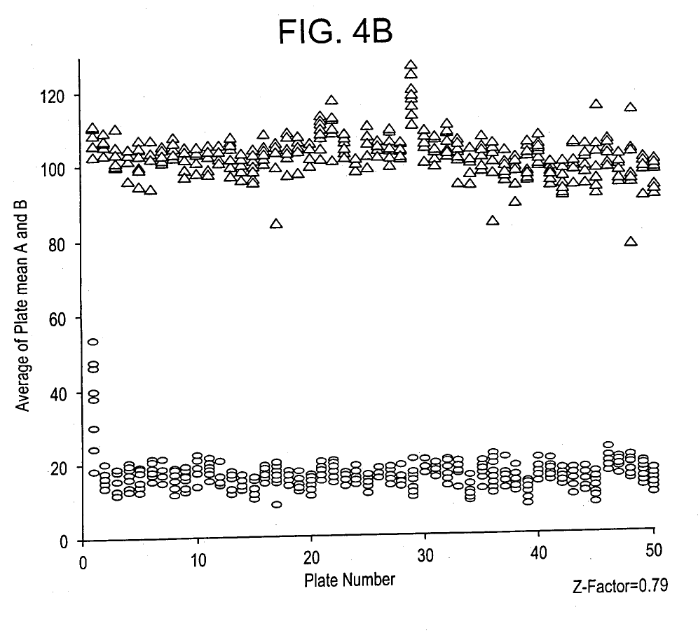

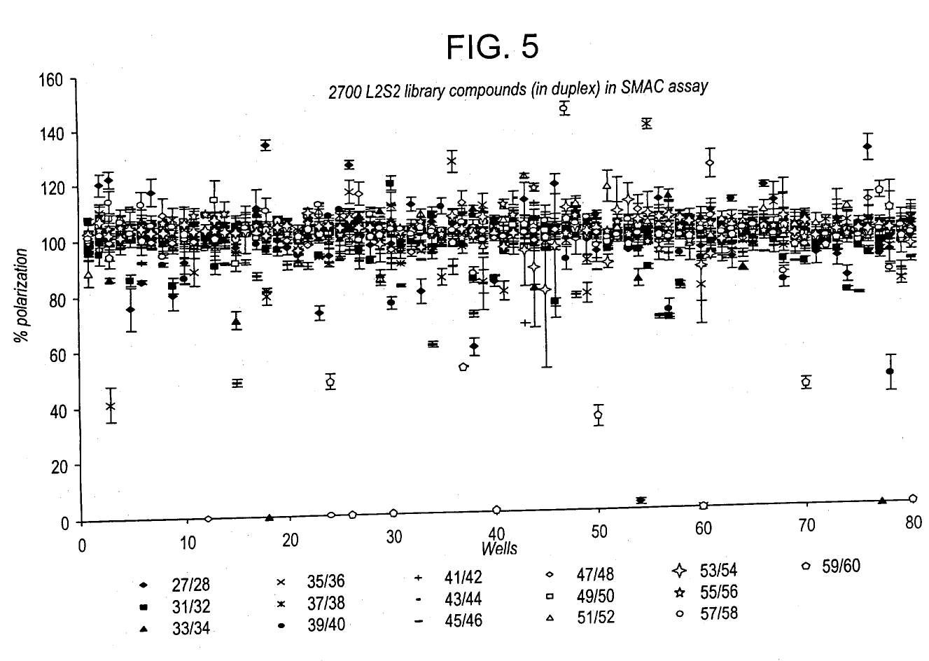

We developed an assay to screen for compounds capable of competing for the binding of an IAP-interacting peptide and displacing the peptide from its binding groove on BIR3 of XIAP (FIGS. 2-5). The BIR3 domain was produced as a GST fusion protein in E. Coli, as the C-terminal portion of XIAP lacking BIR1 and BIR2, and containing BIR3 and the RING domain to the final C-terminal residue, and purified to greater than 95% purity. Only the properly folded BIR3 domain is required here, and many fusion and purification strategies are available to achieve this.

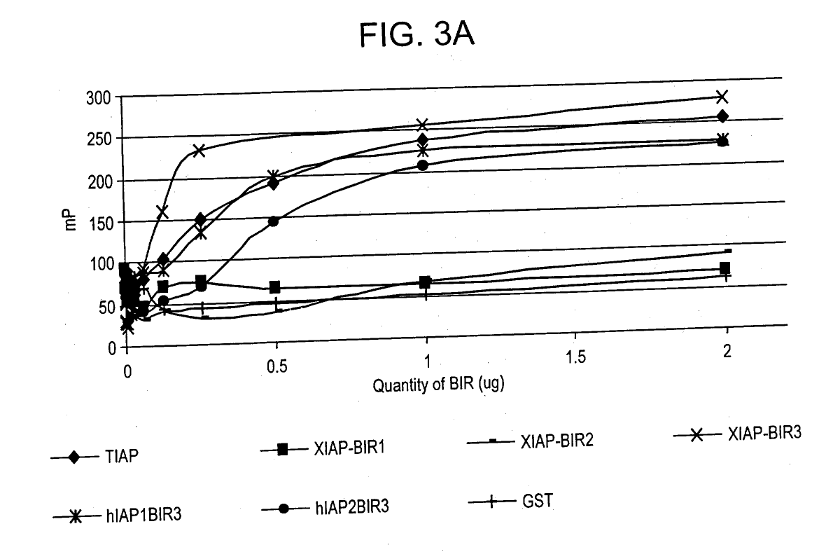

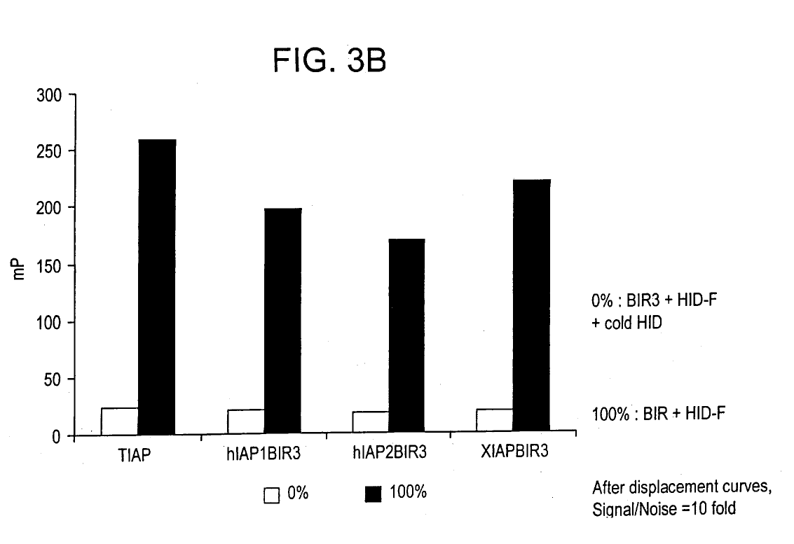

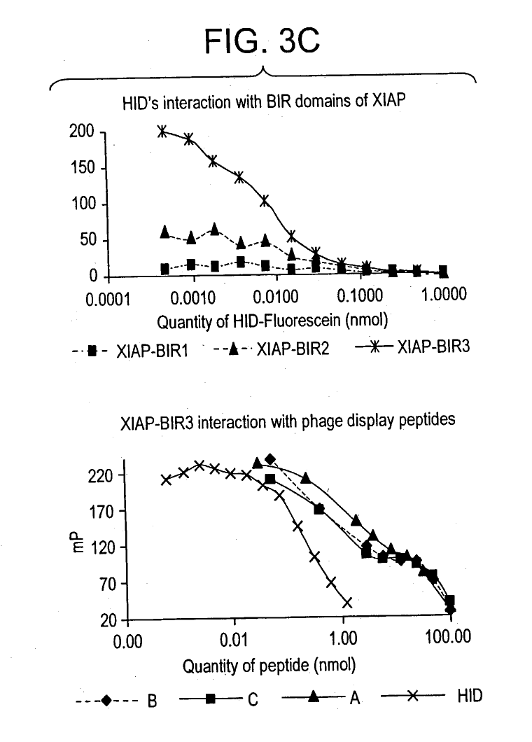

We used a seven amino acid residue peptide based on Drosophila HID (Ala-Val-Pro-Phe-Tyr-Leu-Pro-Gly-Gly (Seq ID NO: 84; a Gly-Gly spacer is included for the purposes of coupling to fluorescein), as it had previously been shown that this bound with high affinity to XIAP BIR3 (FIGS. 3A and 3B). The peptide was produced by standard peptide chemistry (orthogonal synthesis) approaches with a C-terminal fluorescein group as a probe for the fluorescence polarization (FP). The fluor in this position does not interfere with the XIAP binding.

The FP assay is based on the following principles: a smaller molecule tumbles more rapidly in solution than a larger one. A tumbling fluor will depolarize plane-polarized light, and any change in tumbling rate due to binding of the peptide to a larger molecule such as XIAP or the displacement of the peptide from XIAP, and therefore any change in fluorescence polarization can be accurately and sensitively measured (FIGS. 2A and 2B). The assay provides a good signal-to-noise ratio (over 8-fold), and good linearity over the test range. The assay is specific for detecting those peptides or compounds that bind to XIAP BIR3, as demonstrated by the ability of unlabelled excess HID peptide to compete off the fluorescein-labeled peptide, while an N-terminal acetylated peptide could only displace the FP probe when the acetylated peptide was used at a 100-fold increased concentration over then non-acetylated competitor (FIG. 3B). The FP assay also demonstrated that the novel IAP-interacting peptides identified in the phage display library, synthesized as 10 residue peptides, could also compete for HID peptide (nine residues) binding to XIAP BIR3 under the conditions tested (FIG. 3C). This result demonstrates that the peptides identified by binding to BIR3 in the phage binding assay (and therefore part of a longer phage polypeptide) can also bind to XIAP BIR3 as a short peptide. A screen of a set of approximately 1200 defined small molecule compounds of synthetic drug-like molecules demonstrates the reproducibility and robustness of this assay, with a good z-value of over 0.5.

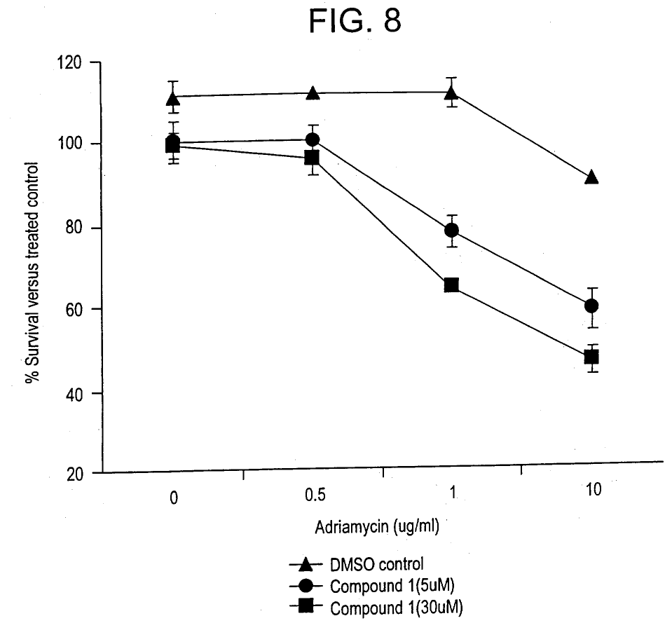

The vast majority of compounds did not displace the labeled probe. Only a small number of compounds could effectively compete off the probe. However, a large proportion of these were false-positives due to intrinsic fluorescence or fluorescence quenching effect of some of the drug-like compounds. This is a well-known problem with FP assays and false positives are easily identified and filtered out. Of the two hits identified in this subset of true positive compounds, one compound (“the compound”) was shown to sensitize cancer cell lines resistant to chemotherapy agents (FIG. 8).

T24 bladder carcinoma cells that are highly resistant to adriamycin were sensitized to this compound and showed a 50% reduction in viability when incubated with 5-30 μM of the compound and 10 μg/ml of adriamycin (a dose which does not result in any cell death on its own), while 5-30 μM of the compound alone did not show any toxicity. The addition of the compound to adriamycin on T24 cells resulted in a shift of several fold of the IC₅₀, such that less adriamycin was required to kill the cancer cells. This is what we would expect from a compound that blocks the ability of IAPs to block caspases once the cell death pathways are activated by a death stimulus such as chemotherapy or radiation. Cancer cells in vivo may also die by the simple addition of the compound if the cancer cell is stressed by oncogene activation, nutrient deprivation, or loss of contact, and the IAPs are blocking caspases from effecting programmed cell death. The compound would be expected to antagonize IAP function and allow caspase activation under these situations.

Having established ideal conditions for high throughput screening in a limited capacity, we then went on to screen larger a larger pool of small molecules. Five different small molecule libraries consisting of natural products and synthetic compounds were screened by the above-described method. In all, 26,800 compounds were screened for their ability to displace the HID-BIR3 interaction. From these screens, over 200 hits were reevaluated, resulting in over 100 candidate compounds. These compounds were tested in IC₅₀ curves against BIR3 with 81 hits producing good IC₅₀ values.

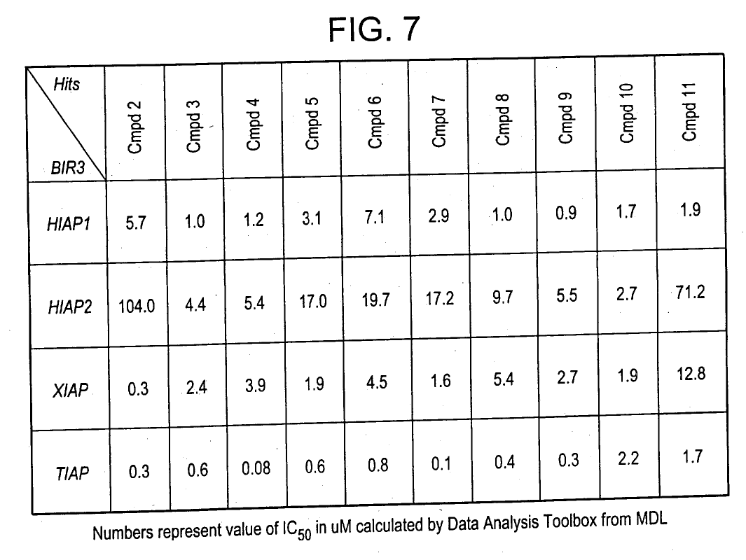

Results, summarized in FIG. 7, show further testing for specific BIR3 domain selectivity. Candidate compounds were tested for their selectivity to HIAP1, HIAP2, and XIAP (FIG. 7). Compounds can be divided in to two groups, pan BIR3-interacting compounds and specific BIR3-interacting compounds.

Experimental Procedures

Materials. XIAP BIR3 domains were expressed as GST-fusion proteins (Pharmacia; Uppsala, Sweden) according to manufacturer's recommendations. Briefly, overnight cultures of E. coli (40 ml) transformed with DNA encoding the GST fusion proteins or just GST alone were diluted 1:40 (1000 ml) in fresh terrific broth supplemented with ampicillin to 100 μg/ml. Cells were grown to OD₆₀₀ ˜0.7, induced with 1 mM IPTG for 3 hours. Cells were harvested and frozen at −80° C. On the next day the cell pellet was thawed and mix with 10 ml of lysis buffer (50 mM Tris pH 7.5, 200 mM NaCl, 1 mM DTT, 1 mM PMF, 20 mg of lysosyme added fresh). Cells were lysed by five passages of the cell suspension in a Bioneb cell-disruptor apparatus (Glas-Col; Terre Haute, Ind.) set at 100 PSI of nitrogen. Lysates were clarified by 20 minutes centrifugation at 4° C. at 20000 g and were then incubated for 1 hour at 4° C. with immobilized glutathione-Sepharose (Pharmacia). The immobilized GST fusion proteins were washed with three column volumes with buffer and eluted with three column volumes of buffer containing 10 mM reduced glutathione. Eluted samples were pooled and an aliquot resolved on SDS-PAGE and analyzed for purity. Pooled purified proteins were then precipitated with ammonium sulfate for 40 minutes at 4° C. Upon completed dissolution of the ammonium salt, precipitated proteins were centrifuged at 18000×g for 15 minutes at 4° C., resuspended at a concentration of 2 mg/ml in PBS, aliquoted, and kept frozen at −80° C. until further use.

Peptide synthesis and conjugation. The HID peptide, Fmoc-Ala-Val-Pro-Phe-Tyr(But)-Leu-Pro-Gly(But)-Gly-OH (Seq ID NO: 85) was prepared using standard Fmoc chemistry on 2-chlorotrityl chloride resin (Int. J. Pept. Prot. Res. 38:555-561, 1991). Cleavage from the resin was performed using 20% acetic acid in dichloromehane (DCM), which left the side chain still blocked. Free terminal carboxylate peptide was then coupled to 4′(aminomethy)-fluorescein (Molecular Probes, A-1351; Eugene, Oreg.) using excess diisopropylcarbodiimide (DIC) in dimethylformamide (DMF) at room temperature. The fluorescent N-C blocked peptide was purified by silica gel chromatography (10% methanol in DCM). The N terminal Fmoc group was then removed using piperidine (20%) in DMF, and the N-free peptide, purified by silica gel chromatography (20% methanol in DCM, 0.5% HOAc). Finally, the t-butyl side chain protective groups were removed using 95% trifluoroacetic acid containing 2.5% water and 2.5% triisopropyl silane. The peptide obtained in such a manner gave a single peak by HPLC and was sufficiently pure for carrying on with the assay.

Assays and High Throughput assays. On the day of screening all reagents were diluted at the appropriate concentration and the working solution kept on ice. The working stock concentration for GST and GST fusion proteins were 4 ng/μl, Fluorescein-labeled HID peptides were used at a concentration of 1.56 fmol/μl, while cold peptides were at 25 pmol/μl. Samples were incubated at a total volume of 200 μl per well in black flat bottom plates, Biocoat, #359135 low binding (BD BioSciences; Bedford, Mass.). Assays were started with the successive addition (using a Labsystem Multi-Drop 96/384 device (Labsystem; Franklin, Mass.) of 50 μl test compounds, diluted in 10% DMSO (average concentration of 28 μM), 50 μl of 50 mM MES-pH 6.5, 50 μl of Fluorescein-HID, 50 μl of GST BIR3/Ring). Unlabeled HID peptide (50 μl) was used as negative control. Once added, all the plates were placed at 4° C. Following overnight incubation at 4° C., the fluorescence polarization was measured using a Polarion plate reader (Tecan, Research Triangle Park, N.C.). A Xenon flash lamp equipped with an excitation filter of 485 nm and an emission filter of 535 nm. The number of flashes was set at 30. Raw data were then converted into a percentage of total interaction(s). All further analysis were performed using the Spotfire data analysis software (Spotfire; Somerville, Mass.)

Upon selection of active compounds, auto-fluorescence of the hits was measured as well as the fluorescein quenching effect, where a measurement of 2000 or more units indicated auto-fluorescence, while a measurement of 50 units indicated a quenching effect. Confirmed hits were then run in dose-response curves (IC₅₀) for reconfirmation. Best hits in dose-response curves were then run into the selectivity assays using BIR3 domain from HIAP1, HIAP2, and XIAP (FIG. 7).

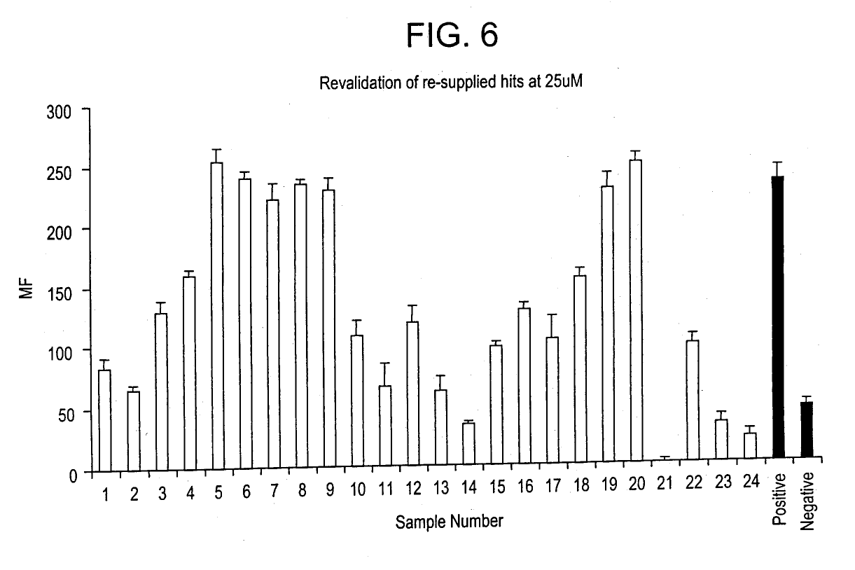

Upon primary screening, hits were re-supplied from Talon Cheminformatics (Acton, ON, Canada) and a new 20 mM drug stock was prepared in 10% DMSO. The HID displacement assay was performed as previously described. Some hits could not be reconfirmed following the re-supply of new lot of compounds. Fluorescence Polarization Units are expressed in MP (FIG. 6).

Alternate binding assays. Fluorescence polarization assays are but one means to measure protein-protein interactions in a screening strategy. Alternate methods for measuring protein interactions may be utilized. Such methods include, but are not limited to mass spectrometry (Neslson and Krone, J. Mol. Recognit., 12:77-93, 1999), surface plasmon resonance (Spiga et al., FEBS Lett., 511:33-35, 2002; Rich and Mizka, J. Mol. Recognit., 14:223-228, 2001; Abrantes et al., Anal. Chem., 73:2828-2835, 2001), fluorescence resonance energy transfer (FRET) (Bader et al., J. Biomol. Screen, 6:255-264,2001; Song et al., Anal. Biochem. 291:133-41, 2001; Brockhoff et al., Cytometry, 44:338-248, 2001), bioluminescence resonance energy transfer (BRET) (Angers et al., Proc. Natl. Acad. Sci. USA, 97:3684-3689, 2000; Xu et al., Proc. Natl. Acad. Sci. USA, 96:151-156, 1999), fluorescence quenching (Engelborghs, Spectrochim. Acta A. Mol. Biomol. Spectrosc., 57:2255-2270, 1999; Geoghegan et al., Bioconjug. Chem. 11:71-77, 2000), fluorescence activated cell scanning/sorting (Barth et al., J. Mol. Biol., 301:751-757, 2000), ELISA, and radioimmunoassay (RIA).

Test extracts and compounds. In general, compounds that affect BIR-peptide interactions are identified from large libraries of both natural products, synthetic (or semi-synthetic) extracts or chemical libraries, according to methods known in the art.

Administration of IAP-Interacting Peptidomimetic Small Molecules

By selectively disrupting or preventing IAPs from binding to their natural partner(s) through its binding site, the peptides of the invention, or derivatives or peptidomimetics thereof, can significantly decrease the ability of an IAP to promote survival of neoplastic cells. Therefore, the peptides of the invention, or derivatives or peptidomimetics thereof, can be used in the treatment of cancer or other neoplasms, when enhanced apoptosis is desired or required.

Cancers and other neoplasms include, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealioma, hemangioblastoma, acoustic neuroma, oligodenroglioma, schwannoma, meningioma, melanoma, neuroblastoma, and retinoblastoma).

A BIR-interacting peptide or peptidomimetic small molecule may be administered within a pharmaceutically-acceptable diluent, carrier, or excipient, in unit dosage form. Conventional pharmaceutical practice may be employed to provide suitable formulations or compositions to administer the compounds to patients suffering from a disease that is caused by excessive cell proliferation.

Administration may begin before the patient is symptomatic. Any appropriate route of administration may be employed, for example, administration may be parenteral, intravenous, intra-arterial, subcutaneous, intramuscular, intracranial, intraorbital, ophthalmic, intraventricular, intracapsular, intraspinal, intracistemal, intraperitoneal, intranasal, aerosol, suppository, or oral administration.

If desired, treatment with a BIR-interacting peptide or small molecule may be combined with more traditional therapies for the proliferative disease such as surgery or administration of chemotherapeutics or other anti-cancer agents, including, for example, γ-radiation, alkylating agents (e.g., nitrogen mustards such as cyclophosphamide, ifosfamide, trofosfamide, and chlorambucil; nitrosoureas such as carmustine, and lomustine; alkylsulphonates such as bisulfan and treosulfan; triazenes such as dacarbazine; platinum-containing compounds such as cisplatin and carboplatin), plant alkaloids (e.g., vincristine, vinblastine, anhydrovinblastine, vindesine, vinorelbine, paclitaxel, and docetaxol), DNA topoisomerase inhibitors (e.g., etoposide, teniposide, topotecan, 9-aminocamptothecin, (campto) irinotecan, and crisnatol), mytomycins (e.g., mytomicin C), antifolates (e.g., methotrexate, trimetrexate, mycophenolic acid, tiazofurin, ribavirin, EICAR, hydroxyurea, and deferoxamine), uracil analogs (5-fluorouracil, floxuridine, doxifluridine, and ratitrexed), cytosine analogs (cytarbine, cytosine arabinoside, and fludarabine), purine analogs (e.g., mercaptopurine, and thioguanine), hormonal therapies (e.g., tamoxifen, raloxifene, megestrol, goserelin, leuprolide acetate, flutamide, and bicalutamide), vitamin D3 analogs (EB 1089, CB 1093, and KH 1060), vertoporfin, phthalocyanine, photosensitizer Pc4, demethoxy-hypocrellin A, interferon-α, interferon-γ, tumor necrosis factor, lovastatin, 1-methyl-4-phenylpyridinium ion, staurosporine, actinomycin D, dactinomycin, bleomycin A2, bleomycin B2, adriamycin, peplomycin, daunorubican, idarubican, epirubican, pirarubican, zorubican, mitoxantrone, and verapamil.

For any of the methods of application described above, the BIR-interacting small molecule may be applied to the site of the needed apoptosis event (for example, by injection), or to tissue in the vicinity of the predicted apoptosis event or to a blood vessel supplying the cells predicted to require enhanced apoptosis.

The dosage of a BIR-interacting small molecule depends on a number of factors, including the size and health of the individual patient, but, generally, between 0.1 mg and 100 mg is administered per day to an adult in any pharmaceutically acceptable formulation. In addition, treatment by any of the approaches described herein may be combined with more traditional therapies.

### Other Embodiments

All publications and patent applications mentioned in this specification, including U.S. Pat. No. 5,919,912, U.S. Pat. Nos. 6,156,535, and 6,133,437 are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure come within known or customary practice within the art to which the invention pertains and may be applied to the essential features hereinbefore set forth.

What is claimed is:

1. A substantially pure polypeptide having a length of less than 100 amino acids and comprising a sequence selected from Seq ID NOs: 3-21, 26-43, 47-65, and 67-75, said polypeptide capable of forming a complex with a polypeptide comprising a BIR domain.

2. The polypeptide of claim 1, wherein said polypeptide has a length of less than 50 amino acids.

3. The polypeptide of claim 2, wherein said polypeptide has a length of less than 30 amino acids.

4. The polypeptide of claim 3, wherein said polypeptide consists of a sequence selected from Seq ID NOs: 3-21,26-43, 47-65, and 67-75.

5. A method for identifying a compound that modulates IAP biological activity, said method comprising the steps of:
a) contacting a first polypeptide having a length of less than 100 amino acids and comprising a sequence selected from Seq ID NOs: 3-21, 26-43, 47-65, and 67-75, and a second polypeptide comprising a BIR domain to form a complex between said first polypeptide and said second polypeptide;
b) contacting said complex with a candidate compound; and
c) measuring the displacement of said first polypeptide from said second polypeptide,
wherein said displacement of said first polypeptide from said second polypeptide indicates that said candidate compound is a compound that modulates IAP biological activity.

6. The method of claim 5, wherein said first polypeptide consists of a sequence selected from Seq ID NOs: 3-21, 26-43, 47-65, and 67-75.

7. The method of claim 5, wherein said second polypeptide is human XIAP, HIAP 1, HIAP2, TsIAP, or Livin, or a BIR domain containing fragment thereof.

8. The method of claim 5, wherein said first polypeptide and/or said second polypeptide is detectably labeled.

9. The method of claim 5, wherein said second polypeptide is immobilized to a solid support matrix.

10. The method of claim 5, wherein said method further comprises the steps of:
d) providing cancer cells overexpressing said second polypeptide;
e) contacting said cancer cells with said candidate compound; and
f) measuring cell death of said cancer cells.

11. The method of claim 10, wherein said contacting step (e) further comprises contacting said cancer cells with a chemotherapeutic agent.

12. The method of claim 11, wherein said chemotherapeutic agent is selected from a group consisting of adriamycin, doxorubicin, daunorubicin, idarubicin, and mitoxantrone.

13. A method for identifying a compound that modulates IAP biological activity, said method comprising the steps of:
a) contacting a first polypeptide having a length of less than 100 amino acids and comprising a sequence selected from Seq ID NOs: 3-21, 26-43, 47-65, and 67-75 and a second polypeptide comprising a BIR domain in the presence of a candidate compound; and
b) measuring binding of said first polypeptide and said second polypeptide, wherein a decrease in the amount of binding relative to the amount of binding of said first polypeptide and said second polypeptide in the absence of said candidate compound indicates that said candidate compound is a compound that modulates IAP biological activity.

14. The method of claim 13, wherein said first polypeptide consists of a sequence selected from Seq ID NOs: 3-21, 26-43, 47-65, and 67-75.

15. The method of claim 13, wherein said second polypeptide is human XIAP, HIAP1, HIAP2, TsIAP, or Livin, or a BIR domain containing fragment thereof.

16. The method of claim 13, wherein said first polypeptide and/or said second polypeptide is detectably labeled.

17. The method of claim 13, wherein said second polypeptide is immobilized to a solid support matrix.

18. The method of claim 13, wherein said method further comprises the steps of:
c) providing cancer cells overexpressing said second polypeptide;
d) contacting said cancer cells with said candidate compound; and
e) measuring cell death of said cancer cells.

19. The method of claim 18, wherein said contacting step (e) further comprises contacting said cancer cells with a chemotherapeutic agent.

20. The method of claim 19, wherein said chemotherapeutic agent is selected from a group consisting of adriamycin, doxorubicin, daunorubicin, idarubicin, and mitoxantrone.
